(19) United States
(12) Patent Application Publication
(10) Pub. No.: US 2021/0047611 A1
(43) Pub. Date: Feb. 18, 2021

(54) MICROWELL PLATE WITH LAMINATED MICRO EMBOSSED FILM BOTTOM

(71) Applicant: StemoniX Inc., Eden Prairie, MN (US)

(72) Inventor: Robert John Petcavich, The Woodlands, TX (US)

(21) Appl. No.: 16/942,415

(22) Filed: Jul. 29, 2020

Related U.S. Application Data
(63) Continuation of application No. 15/753,764, filed on Feb. 20, 2018, now abandoned.
(60) Provisional application No. 62/208,011, filed on Aug. 21, 2015.

(51) Int. Cl. C12N 5/00; B29C 59/04; B29C 35/08

(52) U.S. Cl. CPC C12N 5/0068; B29C 59/04; B29C 35/0805; B29C 2035/0833; C12N 2535/10; B29C 2035/0827; C12N 2533/30

(57) ABSTRACT

The current disclosure describes a method of fabricating a n×n where n=1 to 100 micro well plate that has a transparent film bottom where the film has embossed on the surface micro structures for the facilitation of cell growth and differentiation in particular cardiomyocyte cells derived from human induced pluripotent stem cells. In one embodiment, the micro well plate has an array of 384 locations molded out of thermoplastic onto which a micro embossed film is laminated to the bottom. The micro embossed features are fabricated such that that when the film is laminated to the plate, e.g., with adhesive or via welding, the embossed microstructures are located within the individual microplate well locations.

### CROSS-REFERENCE TO RELATED APPLICATIONS

This application is a continuation of U.S. application Ser. No. 15/753,764, filed Feb. 20, 2018 which is a national stage application filed under 35 U.S.C. § 371 from International Application Serial No. PCT/US2016/047721, filed on Aug. 19, 2016, and published on Mar. 2, 2017 as WO 2017/034954, and claims the benefit of the filing date of U.S. Provisional Application Ser. No. 62/208,011, filed on Aug. 21, 2015, which applications and publication are incorporated by reference herein in their entireties.

### BACKGROUND

A microtiter plate (spelled Microtiter is a registered trade name in the United States) or microplate or microwell plate or multiwell, is a flat plate with multiple “wells” used as small test tubes. The microplate has become a standard tool in analytical research and clinical diagnostic testing laboratories. A very common usage is in the enzyme-linked immunosorbent assay (ELISA), the basis of most modern medical diagnostic testing in humans and animals.

A microplate typically has 6, 24, 96, 384 or even 1536 sample wells arranged in a 2:3 rectangular matrix. Some microplates have even been manufactured with 3456 or even 9600 wells, and an “array tape” product has been developed that provides a continuous strip of microplates embossed on a flexible plastic tape.

Each well of a microplate typically holds somewhere between tens of nanoliters to several milliliters of liquid. They can also be used to store dry powder or as racks to support glass tube inserts. Wells can be either circular or square. For compound storage applications, square wells with close fitting silicone cap-mats may be employed. Microplates can be stored at low temperatures for long periods, may be heated to increase the rate of solvent evaporation from their wells and can even be heat-sealed with foil or clear film. Microplates with an embedded layer of filter material were developed in the early 1980s by several companies, and today, there are microplates for just about every application in life science research which involves filtration, separation, optical detection, storage, reaction mixing, cell culture and detection of antimicrobial activity.

The enormous growth in studies of whole live cells has led to an entirely new range of microplate products which are “tissue culture treated” especially for this work. The surfaces of these products are modified using an oxygen plasma discharge to make their surfaces more hydrophilic so that it becomes easier for adherent cells to grow on the surface which would otherwise be strongly hydrophobic.

A number of companies have developed robots to specifically handle microplates. These robots may be liquid handlers which aspirate or dispense liquid samples from and to these plates, or “plate movers” which transport them between instruments, plate stackers which store microplates during these processes, plate hotels for longer term storage, plate washers for processing plates, plate thermal sealers for applying heat seals, de-sealers for removing heat seals, or microplate incubators to ensure constant temperature during testing. Instrument companies have designed plate readers, which can detect specific biological, chemical or physical events in samples stored in these plates.

Microplates are manufactured in a variety of materials. The most common is polystyrene used for most optical detection microplates. It can be colored white by the addition of titanium dioxide for optical absorbance or luminescence detection or black by the addition of carbon for fluorescent biological assays. Polypropylene is used for the construction of plates subject to wide changes in temperature, such as storage at −80° C. and thermal cycling. It has excellent properties for the long-term storage of novel chemical compounds.

The most common manufacturing process is injection molding, used for polystyrene, polypropylene and cyclo-olefin. However, microplates are made from a variety of polymers to resist a wide range of temperatures and chemicals. There are also plates made from glass, and or a combination of plastic and with a transparent glass or polymer film bottom used for optical fluorescent studies.

### SUMMARY

With the discovery of iPS cells and their differentiation into various cells types based on not only chemical signals but also surface topography and substrate mechanical properties, it is desirable to be able to create various micro geometric features into the microplate wells.

The present disclosure describes a method to fabricate micro embossed polymer films that can be attached to microplates for the deposition of cells including, for example, iPS-differentiated cells and in one embodiment iPS-differentiated cardiomyocytes, to the film surface that has been attached to a n×n microplate. The micro embossed polymer films provide for self-alignment of iPScs or cells differentiated from iPScs, e.g., cardiomyocytes, neurons, or hepatocytes, into mature like, e.g., adult, human organ or tissue structures. Thus, the patterns in the microstructured film induces cells to be more biologically relevant as found in adult tissue structures. In one embodiment, the film is formed of acrylic, polycarbonate or polystyrene. In one embodiment, the Pascal value may be from 0.5 to 4 GigaPascals (GPas), e.g., from 2 to 3 GPas.

Thus, the presence of microstructures on film lining the receptacles (wells) of the polymer film, e.g., microstructures on at least the bottom of the liner, induces stem cells, or cells differentiated from stem cells, to self-align. In one embodiment, the microstructures form a linear pattern, e.g., to induce self-alignment of cardiomyocytes. In one embodiment, the microstructures form a pattern that is a star or daisy pedal shape, e.g., for self-alignment of hepatocytes. In one embodiment, the microstructures form a pattern that is an undulated serpentine shape, e.g., for self-alignment of neurons. In one embodiment, the pattern features sizes and shape sizes from 0.1 to 200 microns, e.g., from 10 to 100 microns.

In one embodiment, a method to prepare a substrate for culturing cells is provided. In one embodiment, the method includes providing a polymer substrate having a plurality of receptacles for cells in a geometric pattern; and applying to the polymer substrate a polymer film having microstructures in a micropattern that facilitates cell growth and/or differentiation or manufacturing a polymer film having microstructures in a micropattern that facilitate cell growth and/or differentiation. In one embodiment, the microstructures have a height (h) in the range from about 0.1 micron to about 500 microns. In one embodiment, the microstructures have a width (w) from about 1 to about 800 microns and/or a length (l) from about 5 microns to about 75 millimeters. In one embodiment, the microstructures form a linear pattern. In one embodiment, the microstructures form a star or daisy pedal shape. In one embodiment, the microstructures form an undulating serpentine shape. In one embodiment, the microstructures are in a pattern having shape sizes from 0.1 to 200 microns or from 10 to 100 microns. In one embodiment, the modulus of the microstructures is from about 100 Pascal's to about 2.5 GPascal's. In one embodiment, the modulus of the microstructures is from about 0.5 to 4 GPas. In one embodiment, the modulus is from about 2 to 3 GPas. In one embodiment, the microstructures in each receptacle in the film are the same. In one embodiment, the microstructures in different receptacles in the film are different. In one embodiment, the film comprises acrylic, polycarbonate or polystyrene.

Also provided is a system to prepare the substrate having the film with the micropatterned microstructures. In one embodiment, the system is a roll to roll system. In one embodiment, the system includes one or more of a coating module, drying module or embossing module.

In one embodiment, a method of using a substrate micropatterned with microstructures for culturing cells is provided. The method includes providing a polymer substrate having a plurality of receptacles for cells in a geometric pattern, at least one surface of the plurality of receptacles having a polymer film disposed thereon, which polymer film has microstructures in a micropattern that facilitate cell growth and/or differentiation; and introducing and culturing cells in one or more of the plurality of receptacles having the polymer film. In one embodiment, the cells are stem cells, e.g., iPScs. In one embodiment, the cells are cardiomyocytes, neurons, or hepatocytes, e.g., differentiated from iPScs. In one embodiment, the microstructures have a height (h) in the range from about 0.1 micron to about 500 microns. In one embodiment, the microstructures have a width (w) from about 1 to about 800 microns and/or a length (1) from about 5 microns to about 75 millimeters. In one embodiment, the microstructures form a linear pattern. In one embodiment, the microstructures form a star or daisy pedal shape. In one embodiment, the microstructures an undulating serpentine shape. In one embodiment, the microstructures are in a pattern having shape sizes from 0.1 to 200 microns or from 10 to 100 microns. In one embodiment, the modulus of the microstructures is from about 100 Pascal's to about 2.5 GPascal's. In one embodiment, the modulus of the microstructures is from about 0.5 to 4 GPas. In one embodiment, the modulus is from about 2 to 3 GPas. In one embodiment, the microstructures in the film in each receptacle are the same. In one embodiment, the microstructures in the film in different receptacles are different. In one embodiment, the film comprises acrylic, polycarbonate or polystyrene.

### BRIEF DESCRIPTION OF THE DRAWINGS

FIG. 1 illustrates a cross sectional view of a micro patterned film in accordance one embodiment.

FIG. 2 illustrates a top view of a microwell plate with various geometric dimensions.

FIG. 3 illustrates a cross sectional view of the process used to laminate micro embossed films to a microwell plate.

FIG. 4 illustrates a cross sectional view of the process used to manufacture micro embossed films described in one embodiment.

### DETAILED DESCRIPTION

One or more embodiments will be described below. These described embodiments are only exemplary. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure relates generally to providing surfaces with microstructures to enable differentiated cells such as differentiated human iPS cells to attach and grow in microwell plates. More specifically, the disclosure relates to providing various shapes and distributions of microstructures that enable human cells to mimic human organ structures on the micro scale inside individual micro container cells.

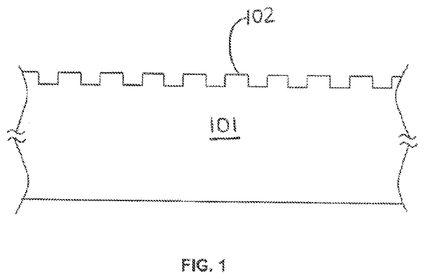

The various embodiments provide a plurality of microstructures on a surface of a substrate to facilitate the attachment and viability of differentiated iPS cells into various human organ tissues. In one embodiment, a plurality of microstructures 102 is formed directly on a surface of a substrate 101, as illustrated in FIG. 1, in order to provide a platform for cell growth. The plurality of microstructures 102 refers to the raised portions of the substrate surface. In another embodiment, the microstructures 102 may be formed on a first surface of a substrate comprising a transparent or translucent glass or polymeric sheet (or film). In some embodiments, a conformal biocompatible layer such as gelatin, fibronectin, polylysine, hyaluronic acid, and collagen may cover the microstructures, the coating provides enhanced cell binding.

Some embodiments provide a variety of microstructure shapes and distributions (e.g., patterns) of microstructures on a surface of a substrate in order to provide a platform for subsequent cellular binding to the surface. In some embodiments, the exterior surface of the substrate or protective layer may have a surface energy in the range from about 1 to about 50 dynes/cm.sup.2 to enhance the spreading of deposited cellular solutions. Furthermore, in some embodiments the density and distribution of microstructures on a protective layer are also optimized in order to maximize cell growth and differentiation. The microstructures may have essentially any geometry.

The microstructures may have a height (h) in the range from about 1 micron to about 250 microns, and in one embodiment in a range from about 50 microns to about 150 microns. The height of the microstructure may be in accordance with the particular applications in terms of the particular cells to be attached and amount of the cells deposited. The width (w) of the microstructures can range from 10 to 500 microns and the length (1) from 10 microns to 25 millimeters.

In another aspect, microstructure embossed geometry may have the requisite mechanical modulus. For example, the modulus can be varied from 100 Pascal's to 2.5 GPascal's that simulate various body tissue mechanical properties such as fatty tissue to bone.

The substrate may comprise any material that may be processed to form a plurality of microstructures (e.g., cylindrical, pyramidal frustum, linear, rectangular or curved elongated microstructure) in a surface of the substrate or protective layer. Suitable substrate materials include glass, metal, and polymer. The plurality of microstructures may be formed into or onto a surface of a substrate by any known processing technique. For example, a planar surface of a glass substrate may be patterned and etched to remove glass material such that a plurality of microstructures are formed and remain on the surface of the substrate. In another example, a surface of a metal substrate (e.g., a metal sheet) may be etched, embossed, or stamped to form microstructures on the surface of the substrate. In yet another example, a polymerizable material on a substrate may be molded, cured by actinic radiation, thermally formed, embossed, ablated, etched, or any of a number of polymer processing techniques to form the microstructures on the surface of the substrate.

Thus, the plurality of microstructures formed in or on a surface of a substrate may comprise the same material as the substrate itself. In other words, the plurality of microstructures formed on a transparent or translucent substrate (e.g., optically clear glass or plastic substrate may be transparent/translucent microstructures that maintain a transmissive property of the substrate surface. Similarly, the plurality of microstructures formed on a nontransparent substrate (e.g., opaque plastic, glass, or metal substrate) may be opaque microstructures.

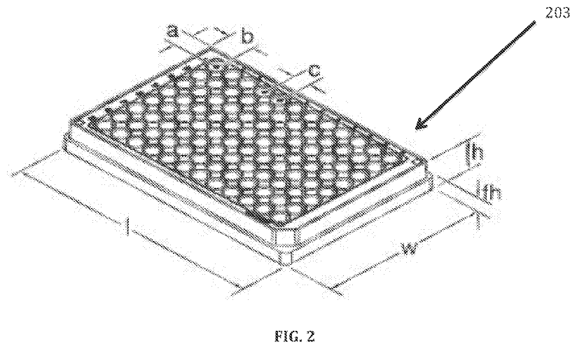

FIG. 2 shows a top view of a typical microwell plate. It is comprised of n×n wells where n can range from, for example, 1 to 100, having microwell dimensions a and b with a well-to-well pitch of c. The length 1, width, w and height h can vary depending on the number of wells and instrumentation compatibility.

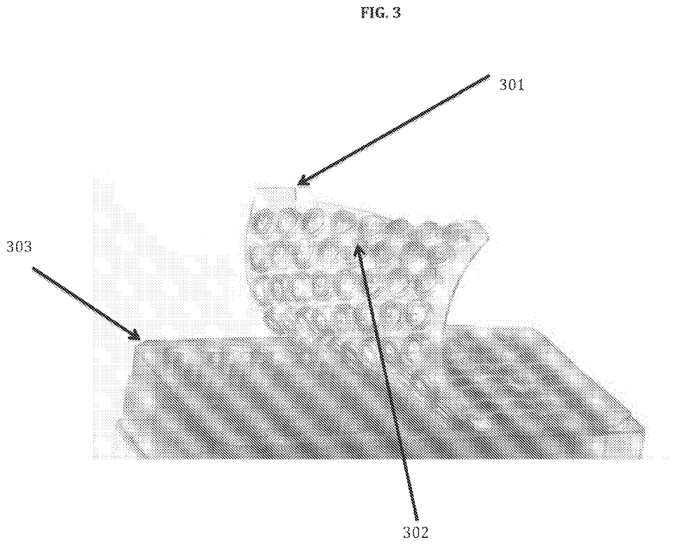

FIG. 3 shows a typical microwell plate 303 onto which a micro embossed film 301, with embossed microstructures 302 being laminated to the plate. The film may be laminated with adhesive or by ultrasonic, laser or thermal welding to the microwell individual well spacing structural elements.

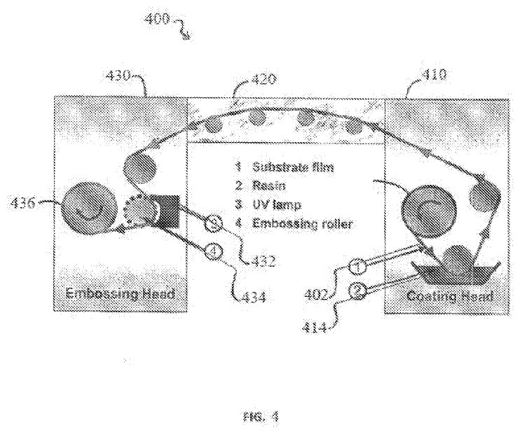

FIG. 4 illustrates an example roll to roll embossing system 400 for manufacturing a substrate 402 having a plurality of microstructures (e.g., such as the microstructures discussed in the descriptions of FIGS. 1-3) distributed on a top surface of the substrate 402. In some implementations, the system 400 may be used to manufacture elongated sheets or rolls of micro patterned substrate.

The system 400 includes a coating module 410, a drying module 420, and an embossing module 430. The coating module 410 accepts a roll 412 of unpatterned substrate 402 (e.g., polyethylene terephthalate film (PET) film). In some embodiments, the roll 412 of unpatterned substrate 402 may be replaced by another form or supply of unpatterned substrate 402 for coating. For example, unpatterned substrate 402 may be supplied as flat sheets, in which case a sheet feeder mechanism may be implemented. In another example, unpatterned substrate 402 may be supplied in fanfold form (e.g., like computer paper), wherein the substrate 402 is presented as substantially flat sheets that are periodically folded to form a zigzag pattern.

The coating module 410 includes a supply of a resin 414 (e.g., ultraviolet curable acrylate) that is applied to the substrate 402. In some implementations, the substrate 402 may be cleaned prior to the application of the resin 414. The resin 414 may be applied in a variety of ways. For example, the substrate 402 may be passed through, or be dipped in a bath of the resin 414, thereby coating the substrate. In other implementations, the resin 414 may be sprayed, rolled, brushed, or otherwise deposited onto the substrate 402.

The substrate 402 passes through the drying module 420. In some implementations, the drying module 420 can dry or partially dry, heat, cure, or otherwise process the resin 414 that was previously applied to the substrate 402 by exposing the substrate 402 to heat or ultraviolet (UV) radiation. In some implementations, by at least partly drying or curing the resin 414, it may become bonded to the substrate 402.

An embossing module 430 processes the substrate 402. The embossing module 430 includes an ultraviolet (UV) lamp 432 and an embossing roller 434. In some implementations, the embossing roller 434 is sleeved by a master shim covered by an inverted (e.g., negative) pattern of microstructures such as the microstructures previously discussed in the descriptions of FIGS. 1-4. In some embodiments, the inverse pattern of microstructures may be formed using a photolithographic process. For example, a master shim's substrate may be cleaned and coated with a photoresist material, and may then be pre-cured by baking or exposure to UV light. The desired microstructure pattern may then be transferred onto pre-cured photoresist by using a projected image or an optical mask. The photoresist can be developed (e.g., etched) by standard photolithography techniques to form a patterned resist of the desired microstructures, after which the patterned resist can be post-cured. The patterned photoresist material can then be coated with a metal (e.g., copper) to make the surface conductive, and then nickel can be electroplated onto the metal-coated patterned resist thereby forming a nickel master shim. The nickel master shim can then be separated from the substrate so it can be wrapped around a drum to form the embossing roller 434.

The embossing roller 434 is brought into rolling contact with the resin 414 coating on the substrate 402. As the embossing roller 434 rolls over the substrate 402, the inverted pattern of microstructures is impressed into the resin harden, thereby preserving the patterns of microstructures impressed into the resin 414. The substrate 402 may be molded, thermally formed, embossed, etched, or otherwise be patterned using any of a number of polymer processing techniques to form the microstructures on a surface of the protective layer. A roll 436 takes up the substrate 402. In some implementations, the roll 436 can be replaced by a receptacle for separated sheets, fan-folded sheets, or other forms of the substrate 402 after processing. In some implementations, once the substrate 402 has been processed, an adhesive and a protective liner can be applied to the smooth (e.g., unpatterned) side of the substrate 402. In some implementations, the substrate 402 can be cut to a desired size. For example, the substrate 402 can be cut into pieces that are subsequently laminated with adhesive or welded to the bottom of a microwell plate.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. (canceled)

2. A method of using a substrate micropatterned with microstructures for culturing cells, comprising:
providing a polymer substrate having a microembossed polymer film having a plurality of receptacles for cells in a geometric pattern, which polymer film comprises acrylic, polycarbonate or polystyrene and has microstructures in a micropattern that facilitate cell growth and/or differentiation, wherein the microstructures form a linear pattern, a star or daisy petal shape, or an undulating serpentine shape; and
culturing human cells in one or more of the plurality of receptacles having the polymer film, thereby facilitating cell growth and/or differentiation of the human cells, wherein the human cells are cardiomyocytes, neurons, or hepatocytes or stem cells that differentiate to cardiomyocytes, neurons or hepatocytes.

3. The method of claim 2 wherein the human cells are the stem cells or the cardiomyocytes.

4. The method of claim 2 wherein the microstructures have a height (h) in the range from about 0.1 micron to about 500 microns.

5. The method of claim 2 wherein the microstructures have a width (w) from about 1 to about 800 microns and/or a length (l) from about 5 microns to about 75 millimeters.

6. The method of claim 2 wherein the microstructures form a linear pattern and the cells are cardiomyocytes or human stem cells that differentiate to cardiomyocytes.

7. The method of claim 2 wherein the microstructures are in a pattern having shape sizes from 0.1 to 200 microns or from 10 to 100 microns.

8. The method of claim 2 wherein the modulus of the microstructures is from about 100 Pascal's to about 2.5 GPascal's.

9. The method of claim 2 wherein the modulus of the microstructures is from about 0.5 to 4 GPas.

10. The method of claim 9 wherein the modulus is from about 2 to 3 GPas.

11. The method of claim 2 wherein the microstructures in a receptacle are the same.

12. The method of claim 2 wherein the microstructures form a star or daisy petal shape and the cells are hepatocytes or human stem cells that differentiate to hepatocytes.

13. The method of claim 2 wherein the microstructures form an undulating serpentine shape and the cells are neurons or human stem cells that differentiate to neurons.

14. The method of claim 2 wherein the cells are induced pluripotent stem cells.

15. The method of claim 2 wherein the microstructures have a height (h) in the range from about 50 microns to about 150 microns or about 1 to about 250 microns.

16. The method of claim 2 wherein the film comprises polycarbonate or polystyrene.
